(19) United States
(12) Patent Application Publication
(10) Pub. No.: US 2026/0027040 A1
(43) Pub. Date: Jan. 29, 2026

(54) METHOD OF USING AQUEOUS SOLUTION OF PROPYLENE GLYCOL TO INHIBIT DEGRADATION OF DALBAVANCIN HYDROCHLORIDE, DALBAVANCIN INJECTION FORMULATION, PREPARATION METHOD AND METHOD OF USING THE SAME

(71) Applicant: Kindos Pharmaceuticals Co., Ltd., Chengdu (CN)

(72) Inventors: Yaya Wen, Chengdu (CN); Xuanyu Shi, Chengdu (CN); Qingguo Song, Chengdu (CN)

(21) Appl. No.: 18/898,934

(22) Filed: Sep. 27, 2024

(30) Foreign Application Priority Data
Jul. 26, 2024 (CN) 202411017036.0
Sep. 19, 2024 (CN) 202411310699.1

(51) Int. Cl. A61K 9/00; A61K 9/08; A61K 38/14; A61K 47/10

(52) U.S. Cl. CPC A61K 9/0019; A61K 9/08; A61K 38/14; A61K 47/10

(57) ABSTRACT

A method of using an aqueous solution of propylene glycol to inhibit degradation of dalbavancin hydrochloride, a dalbavancin injection formulation, a preparation method and a method of using the same are provided. In the method of using the aqueous solution of propylene glycol to inhibit degradation of dalbavancin hydrochloride, the volume ratio of propylene glycol to water for injection in the aqueous solution of propylene glycol is 2:8-6:4. In the dalbavancin injection formulation, the concentration of dalbavancin hydrochloride is 20-120 mg/mL, calculated as dalbavancin free base.

### CROSS REFERENCE TO RELATED APPLICATIONS

The present disclosure claims the priority to a Chinese patent application CN2024110170360 filed with the Chinese Patent Office on Jul. 26, 2024, and entitled “USE OF AQUEOUS SOLUTION OF PROPYLENE GLYCOL IN INHIBITING DEGRADATION OF DALBAVANCIN HYDROCHLORIDE, DALBAVANCIN INJECTION FORMULATION, AND PREPARATION METHOD AND USE” and a Chinese patent application CN2024113106991 filed with the Chinese Patent Office on Sep. 19, 2024, and entitled “USE OF AQUEOUS SOLUTION OF PROPYLENE GLYCOL IN INHIBITING DEGRADATION OF DALBAVANCIN HYDROCHLORIDE, DALBAVANCIN INJECTION FORMULATION, AND PREPARATION METHOD AND USE”, the contents of which are incorporated herein by reference in entirety.

### TECHNICAL FIELD

The present disclosure relates to the technical field of pharmaceutical formulations, and specifically to use of an aqueous solution of propylene glycol in inhibiting degradation of dalbavancin hydrochloride, a method of using an aqueous solution of propylene glycol, a dalbavancin injection formulation, a preparation method and use, and a method of using the same.

### BACKGROUND ART

Only innovator of dalbavancin formulation is available in the US market at present, of which a dosage form of product is lyophilized powder of 500 mg per vial. Dalbavancin for injection held by ABBVIE INC was approved by the U.S. Food and Drug Administration (FDA) on May 23, 2014, with a trade name DALVANCE®. It was approved for the treatment of adult and pediatric patients with acute bacterial skin and skin structure infections (ABSSSI) caused by designated susceptible strains of the following Gram-positive microorganisms: Staphylococcus aureus (including methicillin-susceptible and methicillin-resistant isolates), Streptococcus pyogenes, Streptococcus agalactiae, Streptococcus dysgalactiae, Streptococcus anginosus group (including S. anginosus, S. intermedius, S. constellatus) and Enterococcus faecalis (vancomycin susceptible isolates).

DALVANCE® is a sterile, lyophilized, preservative-free, white to off-white to pale yellow solid. Each vial contains dalbavancin HCl (dalbavancin hydrochloride) equivalent to 500 mg of dalbavancin as the free base, plus lactose monohydrate (129 mg) and mannitol (129 mg) as excipients. Sodium hydroxide or hydrochloric acid may be added to adjust the pH at the time of manufacture. The route of administration of DALVANCE® is intravenous infusion, and according to the product package insert, the product needs to be reconstituted and further diluted before intravenous infusion, that is: each vial of product must be first reconstituted with 25 mL of either Sterile Water for Injection or 5% Dextrose Injection. To avoid foaming, alternate between gentle swirling and inversion of the vial until its contents are completely dissolved. Do not shake. The reconstituted vial contains 20 mg/mL dalbavancin as a clear, colorless to yellow solution, which then is further diluted only with the 5% Dextrose Injection to a solution at a final concentration of 1 mg/mL-5 mg/mL. The reconstituted solution and the diluted solution can be refrigerated at 2-8° C. or stored at a controlled room temperature, and the total time from reconstitution to dilution to administration should not exceed 48 hours.

DALVANCE® is a lyophilized formulation with a high production cost, and requires operations of reconstitution and dilution before being used, thus greatly reducing the convenience of administration. Moreover, before clinical use, 25 mL of a diluent needs to be added for reconstitution, to form a 20 mg/mL solution, which is then further diluted for clinical use. Therefore, 50 mL large vials are used for packaging, which increases costs of storage and transportation of the drug.

### SUMMARY

The present disclosure provides use of an aqueous solution of propylene glycol in inhibiting degradation of dalbavancin hydrochloride.

The present disclosure further provides a method of using an aqueous solution of propylene glycol, including using the aqueous solution of propylene glycol to inhibit degradation of dalbavancin hydrochloride.

The present disclosure further provides a dalbavancin injection formulation, containing dalbavancin hydrochloride and an aqueous solution of propylene glycol, wherein the aqueous solution of propylene glycol can inhibit degradation of dalbavancin hydrochloride, wherein a concentration of dalbavancin hydrochloride is 20-120 mg/mL, calculated as dalbavancin free base, and pH of the dalbavancin injection formulation is 3.5-5.5.

The present disclosure further provides a preparation method for the preceding dalbavancin injection formulation, including mixing the aqueous solution of propylene glycol with dalbavancin hydrochloride to obtain a clear mixed solution, and adjusting pH of the mixed solution using a pH regulator to obtain an in-process product bulk solution of dalbavancin injection.

The present disclosure further provides use of the preceding dalbavancin injection formulation in the preparation of a drug for treating bacterial infections.

The present disclosure further provides a method of using the preceding dalbavancin injection formulation, including using the dalbavancin injection formulation in the preparation of a drug for treating bacterial infections.

### DETAILED DESCRIPTION OF EMBODIMENTS

In order to make the objectives, technical solutions and advantages of the examples of the present disclosure clearer, the technical solutions in the examples of the present disclosure will be clearly and completely described below. Examples, for which no specific conditions are specified, are performed according to conventional conditions or conditions recommended by the manufacturers. Where manufacturers of reagents or apparatuses used are not specified, they are conventional products commercially available.

The active pharmaceutical ingredient, dalbavancin hydrochloride, is a heat-sensitive material, hygroscopic, relatively stable under an acidic condition, and prone to degradation under alkaline and high-temperature conditions to generate degradation impurities. In order to inhibit the generation of impurities, dalbavancin hydrochloride is required to be stored under a condition of −20±5° C. According to researches, prominent degradation impurities of dalbavancin hydrochloride are compounds of following Formula I, Formula III and Formula IV.

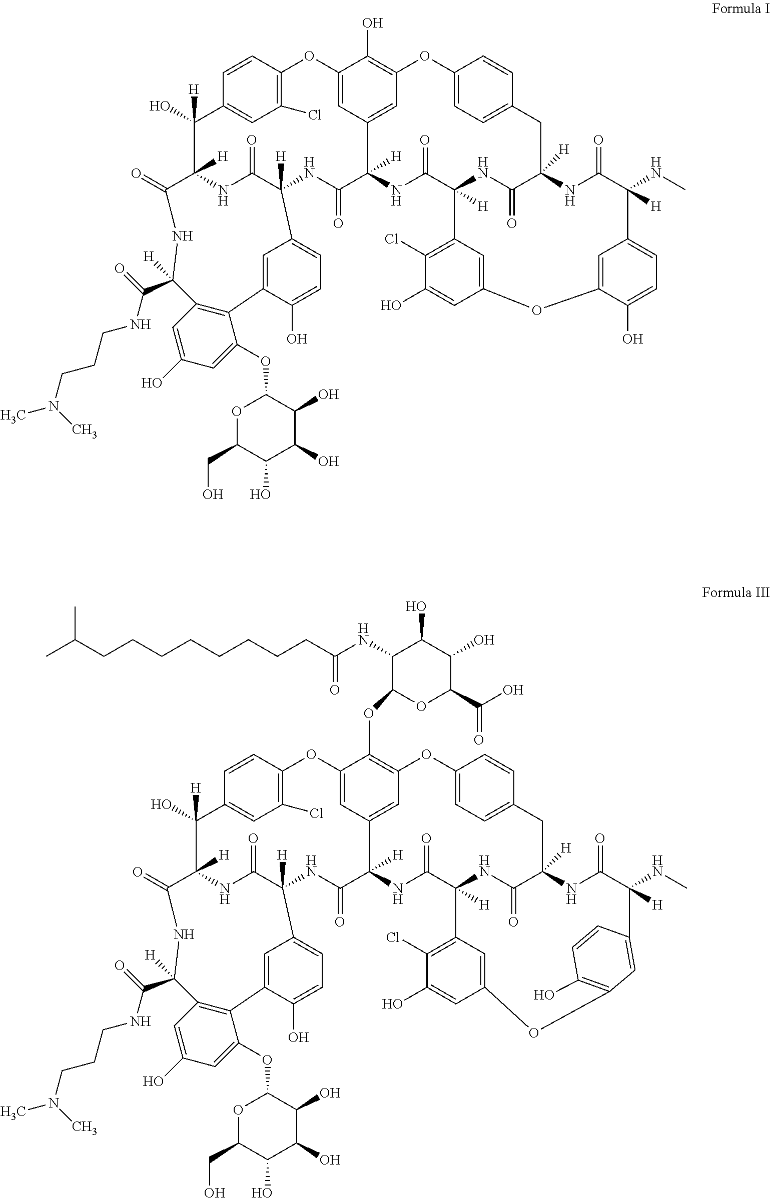

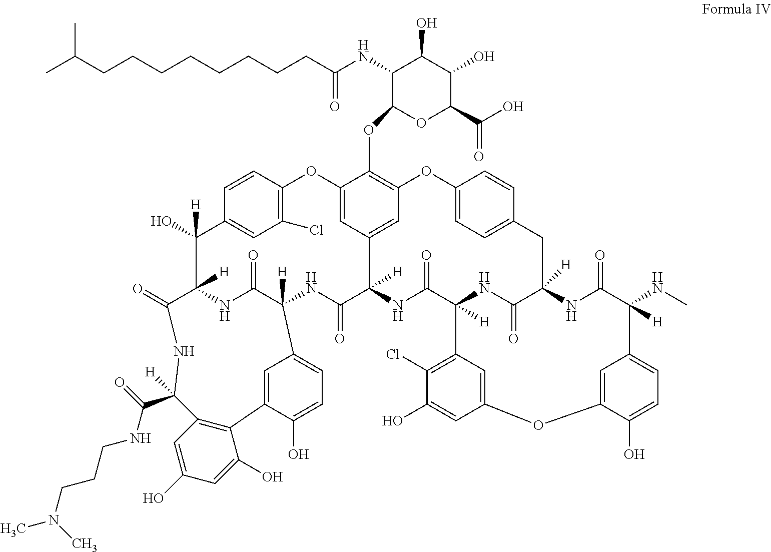

As dalbavancin hydrochloride is prone to degradation in an aqueous-solution state, it causes a high impurity level. Therefore, DALVANCE® prepares the present product into a lyophilized powder, which, before clinical use, must be reconstituted with either Sterile Water for Injection or a 5% Dextrose Injection, and then diluted only with the 5% Dextrose Injection to a solution at a final concentration of 1 mg/mL-5 mg/mL. The lyophilized formulation requires operations of reconstitution and dilution before being used, and in order not to affect the quality of product, storage time of the reconstituted and diluted solutions is very short, greatly reducing the convenience of administration.

In order to solve the above technical problems, for example, to improve the convenience of administration, storage and transportation, the present disclosure provides use of an aqueous solution of propylene glycol in inhibiting degradation of dalbavancin hydrochloride, a method of using an aqueous solution of propylene glycol, a dalbavancin injection formulation, a preparation method, use and a method of using the same.

The present disclosure can overcome the problem that the existing DALVANCE® lyophilized formulation needs to be first reconstituted before being used, and facilitate improving the convenience of administration, storage and transportation.

Embodiments of the present disclosure provide use of an aqueous solution of propylene glycol in inhibiting degradation of dalbavancin hydrochloride.

The embodiments of the present disclosure provide a method of using an aqueous solution of propylene glycol, including using the aqueous solution of propylene glycol to inhibit degradation of dalbavancin hydrochloride.

In the embodiments of the present disclosure, the aqueous solution of propylene glycol is used to inhibit the degradation of dalbavancin hydrochloride, improve the stability of dalbavancin, and facilitate preparation of a dalbavancin injection. Compared with the lyophilized formulation, on one hand, an aqueous injection product can exclude the reconstitution operation and facilitate improving the convenience of administration; and on the other hand, the production process is simpler, the production cycle is shorter, and expensive equipment such as a lyophilizer is not required; therefore, the production cost will be greatly reduced. The great reduction of production cost directly affects the selling price of finished products and also benefits more patients.

In one or more embodiments, in the aqueous solution of propylene glycol, a volume ratio of propylene glycol to water for injection is 2:8-6:4, preferably 4:6-6:4, and more preferably 5.5:4.5-4.5:5.5.

In one or more embodiments, in the aqueous solution of propylene glycol, a volume ratio of propylene glycol to water for injection is preferably 4:6.

In the aqueous solution of propylene glycol, if a proportion of propylene glycol is too large or too small, it is not beneficial to improving the stability of dalbavancin hydrochloride, further leading to increase of total impurities after storage.

The embodiments of the present disclosure further provide a dalbavancin injection formulation, containing dalbavancin hydrochloride and an aqueous solution of propylene glycol, wherein the aqueous solution of propylene glycol can inhibit degradation of dalbavancin hydrochloride, a concentration of dalbavancin hydrochloride is 20-120 mg/mL, calculated as dalbavancin free base, and pH of the dalbavancin injection formulation is 3.5-5.5.

The dalbavancin injection formulation provided by the embodiments of the present disclosure, compared with the existing lyophilized formulation, has the following advantages:

1. The production process is simpler, and the production cost is lower. The production process of the aqueous injection product is simpler, the production cycle is shorter, and the expensive equipment such as the lyophilizer is not required; therefore, the production cost will be greatly reduced. The great reduction of production cost directly affects the selling price of the finished products, and low-price and excellent-quality products will benefit broad masses of patients.

2. Administration is more convenient. The lyophilized formulation needs the reconstitution operation before being used, which reduces the convenience of administration. However, the aqueous injection formulation does not need to be reconstituted before administration, thus reducing operations for administration, improving administration convenience, and meanwhile reducing the risk of introducing microbial contamination by human operations during clinical use of the lyophilized formulation.

3. The safety of product is higher. Successful research and development of the aqueous injection formulation reduces use of lyophilized excipients. Moreover, in the dalbavancin injection formulation of the embodiments of the present disclosure, no additional stabilizer needs to be added, thus reducing types or amounts of excipients in the formulation, so that the safety of the pharmaceutical product is further improved.

4. The product has good stability. The dalbavancin injection formulation of the embodiments of the present disclosure is highly stable, and although a proposed storage condition is 2-8° C. low-temperature storage, it also shows excellent stability under a high-temperature condition (40° C.). After the dalbavancin injection formulation of the embodiments of the present disclosure is placed at 40° C. for 10 days, the impurity level is lower than that of the innovator formulation at near-expiry date under a long-term condition (innovator formulation: expiry date: 2023.01.01, detection date: 2022.04.04, impurity contents: impurity I: 3.29%, impurity III: 1.03%, impurity IV: 0.20%, total impurities: 7.80%). The present disclosure reduces the impurity level and safety risk of the product, and improves product effectiveness.

5. Transportation and storage are more convenient. Before clinical use, the innovator formulation needs to be reconstituted by adding 25 mL of a diluent to form a 20 mg/mL solution, which is further diluted for clinical use; therefore, 50 mL vials are used for packaging. In the present disclosure, a high-concentration dalbavancin injection formulation or a low-concentration dalbavancin injection formulation can be prepared according to requirements, the concentration of dalbavancin in the injection formulation can be up to 120 mg/mL, and in this case, only 5 mL vials need to be used for packaging, which can greatly reduce transportation cost and storage space.

To sum up, the dalbavancin injection formulation of the present disclosure can significantly reduce the production cost and further improve the safety and use convenience of the pharmaceutical product, which not only can bring huge economic benefits to production enterprises, but also can provide more safe, effective and affordable products for broad masses of patients, and the social benefits produced thereby are likewise inestimable.

The active pharmaceutical ingredient used in the present disclosure is in a form of hydrochloride salt of dalbavancin, that is, dalbavancin hydrochloride. Dalbavancin hydrochloride is composed of five closely related active homologues (including A₀, A₁, B₀, B₁ and B₂), where B₀ is the main component of dalbavancin. The homologs share the same core structure, with a structural formula as shown by Formula II:

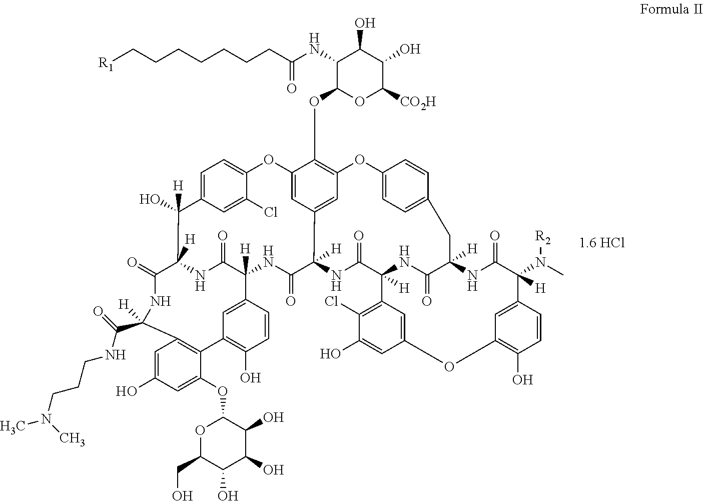

A₀, A₁, B₀, B₁ and B₂ differ in the fatty acid side chain of the N-acylaminoglucuronic acid moiety (R₁) structure and/or the presence of an additional methyl group (R₂) on the terminal amino group. Molecular formulas of A₀, A₁, B₀, B₁ and B₂ and molecular weights of anhydrous free bases corresponding to A₀, A₁, B₀, B₁ and B₂ are as shown in TABLE 1.

TABLE 1

| | | | Molecular | Molecular |
|---|---|---|---|---|
| Dalbavancin | R₁ | R₂ | Formula | Weight |
| A₀ | CH(CH₃)₂ | H | C₈₇H₉₈N₁₀O₂₈Cl₂ · 1.6 HCl | 1802.7 |
| A₁ | CH₂CH₂CH₃ | H | C₈₇H₉₈N₁₀O₂₈Cl₂ · 1.6 HCl | 1802.7 |
| B₀ | CH₂CH(CH₃)₂ | H | C₈₈H₁₀₀N₁₀O₂₈Cl₂ · 1.6 HCl | 1816.7 |
| B₁ | CH₂CH₂CH₂CH₃ | H | C₈₈H₁₀₀N₁₀O₂₈Cl₂ · 1.6 HCl | 1816.7 |
| B₂ | CH₂CH(CH₃)₂ | CH₃ | C₈₉H₁₀₂N₁₀O₂₈Cl₂ · 1.6 HCl | 1830.7 |

Dalbavancin hydrochloride, a white to tan powder, is freely soluble in water, dimethyl sulfoxide and dimethylformamide, very slightly soluble in methanol, and insoluble in chloroform, n-butanol and n-octanol.

In one or more embodiments of the present disclosure, the concentration of dalbavancin in the injection formulation may be, calculated as dalbavancin free base, 20-120 mg/mL, specifically may be 20 mg/mL, 30 mg/mL, 40 mg/mL, 50 mg/mL, 60 mg/mL, 70 mg/mL, 80 mg/mL, 85 mg/mL, 90 mg/mL, 100 mg/mL, 110 mg/mL, 120 mg/mL or any value from 20 mg/mL to 120 mg/mL.

In one or more embodiments, a concentration of dalbavancin hydrochloride may be, calculated as dalbavancin free base, 20-60 mg/mL, preferably 40-60 mg/mL. In one or more embodiments, a concentration of dalbavancin hydrochloride may be, calculated as dalbavancin free base, 60-120 mg/mL, preferably, 60-90 mg/mL, more preferably 80-85 mg/mL. In one or more embodiments, a concentration of dalbavancin hydrochloride may be, calculated as dalbavancin free base, 45-55 mg/mL. If the concentration of dalbavancin is too high and exceeds its solubility in the solvent system, a solid will be precipitated; and if the concentration is too low, on the basis of ensuring an administration effect, an administration volume of the injection is too large, which may cause an increased usage amount of propylene glycol in the formulation, thus increasing the osmolality of the formulation, and affecting administration safety.

In one or more embodiments of the present disclosure, pH of the dalbavancin injection formulation is 3.5-5.5, and specifically may be 3.5, 3.7, 3.9, 4.1, 4.3, 4.5, 4.7, 4.9, 5.1, 5.3, 5.5 or any value from 3.5 to 5.5.

In one or more embodiments of the present disclosure, pH of the dalbavancin injection formulation is 4.0-5.0, preferably 4.4-4.6, more preferably 4.45-4.55. Dalbavancin hydrochloride has better stability under this pH condition, and facilitates reducing the content of impurities in the injection formulation.

After the dalbavancin injection formulation according to the embodiments of the present disclosure is placed at 40° C. for 10 days, at least one of the following conditions is satisfied:
1) the total impurities are less than 5.0%;
2) an increase amount in the content of an impurity as shown by Formula I is less than or equal to 2.5% (≤2.5%), where a compound shown by Formula I is of a structure as follows:

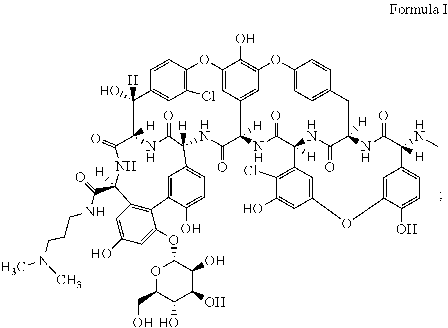

3) a content of the main drug (calculated as the dalbavancin free base) is decreased by 5.0% or less (≤5.0%), wherein the main drug is dalbavancin hydrochloride, including compounds shown by Formula II:

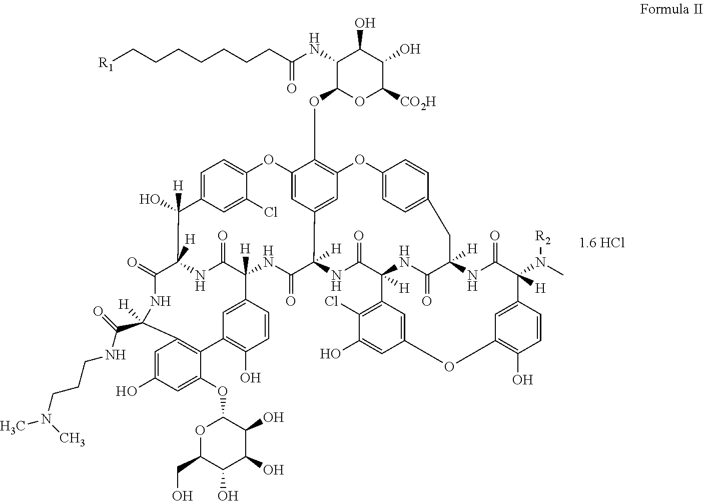

The main drug includes A₀, A₁, B₀, B₁ and B₂, where molecular formulas of A₀, A₁, B₀, B₁ and B₂ and molecular weights of corresponding anhydrous free bases are as shown in TABLE 1 above.

In some embodiments, after the dalbavancin injection formulation is placed at 40° C. for 10 days, the total impurities are less than 5.0%, and the increase in the content of the impurity as shown by Formula I is less than or equal to 2.5% (≤2.5%).

The embodiments of the present disclosure further provide a preparation method for the dalbavancin injection formulation according to any one of the preceding embodiments, including mixing an aqueous solution of propylene glycol with dalbavancin hydrochloride to obtain a clear mixed solution, and adjusting pH of the mixed solution using a pH regulator to obtain an in-process product bulk solution of the dalbavancin injection.

During specific operations, first, about 40% batch size of water for injection is added into a compounding tank, with a solution temperature kept at 15-30° C., a batch required quantity of propylene glycol is added, and mixture is well stirred. A batch required quantity of dalbavancin hydrochloride is added, and stirred until dalbavancin hydrochloride is dissolved to obtain a clear solution, and then pH of bulk solution is adjusted with a pH regulator to 3.5-5.5, wherein the pH regulator is at least one of hydrochloric acid and sodium hydroxide, then the balance amount of water for injection is added to volume, and subsequently the in-process product bulk solution of the dalbavancin injection is sterilization filtered, and filled into clear vials which are then full-stoppered with stoppers, and capped with aluminum caps to obtain finished formulation products.

In the embodiments of the present disclosure, pH of the mixed solution may be adjusted using hydrochloric acid and sodium hydroxide, and only chlorine ions and sodium ions are introduced, thus improving the safety of the injection formulation.

The embodiments of the present disclosure further provide use of the dalbavancin injection formulation according to any one of the preceding embodiments in the preparation of a drug for treating bacterial infections.

The embodiments of the present disclosure further provide a method of using the dalbavancin injection formulation according to any one of the preceding embodiments, including using the dalbavancin injection formulation in the preparation of a drug for treating bacterial infections.

In one or more embodiments, the bacterial infections include acute bacterial skin and skin structure infections (ABSSSI) caused by designated susceptible strains of following Gram-positive microorganisms, including Staphylococcus aureus (including methicillin-susceptible and methicillin-resistant isolates), Streptococcus pyogenes, Streptococcus agalactiae, Streptococcus dysgalactiae, Streptococcus anginosus group (including S. anginosus, S. intermedius, S. constellatus) and Enterococcus faecalis (vancomycin-susceptible isolates).

In one or more embodiments, the drug may be used for the treatment of adult and pediatric patients with acute bacterial skin and skin structure infections (ABSSSI) caused by designated susceptible strains of following Gram-positive microorganisms: Staphylococcus aureus (including methicillin-susceptible and methicillin-resistant isolates), Streptococcus pyogenes, Streptococcus agalactiae, Streptococcus dysgalactiae, Streptococcus anginosus group (including S. anginosus, S. intermedius, S. constellatus) and Enterococcus faecalis (vancomycin-susceptible isolates).

The features and performances of the present disclosure are further described in detail below in conjunction with examples. In the following examples and comparative examples, if not explicitly stated, all contents refer to mass fractions.

### Example 1: Effect of pH on Stability of Dalbavancin Injection Formulation

The present example provides a preparation method for a dalbavancin injection formulation, specifically including the follows.

Into a compounding tank, about 400 mL of water for injection was added, with solution temperature kept at 25° C., a batch required quantity of propylene glycol was added (the volume ratio of propylene glycol to water for injection is 1:1), mixture was well stirred, then a batch required quantity (theoretical required quantity: 50 g, calculated as dalbavancin free base) of dalbavancin hydrochloride was added, and stirred to be dissolved to obtain a clear solution.

The pHs of bulk solutions of formulation 1 to formulation 7 were respectively adjusted to preset values 3.0, 3.5, 4.0, 4.5, 5.0, 5.5 and 5.9 using a pH regulator (sodium hydroxide and/or hydrochloric acid); and then the bulk solutions were made up to 1000 mL with water for injection.

Each bulk solution was filtered through a sterilization filter. About 10.6 mL of the bulk solution was filled into 10 mL clear vial which was then full-stoppered with a 20 mm stopper and capped with an aluminum cap to obtain the finished formulation product. Impurity contents of the finished formulation products were measured when the preparation of the finished formulation products was completed and after the finished formulation products were placed at 40° C. for 10 days, to obtain stabilities of various formulations as shown in TABLE 2.

TABLE 2

| | | | | | Related Substances (%) | | | |
|---|---|---|---|---|---|---|---|---|
| | | | | Assay | Impurity | Impurity | Impurity | Total |
| Formulation | | Appearance | pH | (%) | I | III | IV | Impurities |
| Formulation | Initial | Colorless | 2.98 | 100.9 | 1.003 | 0.038 | 0.073 | 1.571 |
| 1 | | clear solution | | | | | | |
| | 40° C. | Colorless | 2.93 | 96.9 | 3.437 | 0.037 | 0.079 | 4.244 |
| | 10 | clear solution | | | | | | |
| | days | | | | | | | |
| Formulation | Initial | Colorless | 3.40 | 102.0 | 0.999 | 0.038 | 0.067 | 1.561 |
| 2 | | clear solution | | | | | | |
| | 40° C. | Colorless | 3.40 | 97.8 | 3.180 | 0.039 | 0.033 | 3.976 |
| | 10 | clear solution | | | | | | |
| | days | | | | | | | |
| Formulation | Initial | Colorless | 3.91 | 100.8 | 0.983 | 0.039 | 0.064 | 1.542 |
| 3 | | clear solution | | | | | | |
| | 40° C. | Colorless | 3.89 | 98.0 | 2.779 | 0.038 | 0.049 | 3.603 |
| | 10 | clear solution | | | | | | |
| | days | | | | | | | |
| Formulation | Initial | Colorless | 4.36 | 100.4 | 1.001 | 0.040 | 0.063 | 1.564 |
| 4 | | clear solution | | | | | | |
| | 40° C. | Colorless | 4.36 | 97.6 | 2.527 | 0.041 | 0.066 | 3.391 |
| | 10 | clear solution | | | | | | |
| | days | | | | | | | |
| Formulation | Initial | Colorless | 4.91 | 102.1 | 0.992 | 0.039 | 0.060 | 1.554 |
| 5 | | clear solution | | | | | | |
| | 40° C. | Colorless | 4.88 | 96.6 | 2.563 | 0.046 | 0.131 | 3.583 |
| | 10 | clear solution | | | | | | |
| | days | | | | | | | |
| Formulation | Initial | Milky white | N/A¹ | N/A¹ | N/A¹ | | | |
| 6 | | gel-like solid, | | | | | | |
| | | with fluidity1 | | | | | | |
| | 40° C. | Colorless | 5.34 | 97.4 | 2.713 | 0.054 | 0.146 | 3.948 |
| | 10 | clear solution | | | | | | |
| | days | | | | | | | |
| Formulation | Initial | Milky white | N/A¹ | N/A¹ | N/A¹ | | | |
| 7 | | gel-like solid, | | | | | | |
| | | with fluidity1 | | | | | | |
| | 40° C. | Colorless | N/A² | N/A² | N/A² | | | |
| | 10 | clear | | | | | | |
| | days | solution2 | | | | | | |

Notes:
Assay in TABLES 2, 5, 7, 9 and 10 refers to a ratio of the actually measured concentration of dalbavancin hydrochloride (calculated as dalbavancin free base) to a preset concentration of dalbavancin hydrochloride (calculated as dalbavancin free base), where in TABLE 2, the preset concentration of dalbavancin hydrochloride (calculated as dalbavancin free base) for Formulations 1-7 is 50 mg/mL; in TABLE 5, the preset concentration of dalbavancin hydrochloride (calculated as dalbavancin free base) for Formulations 8-10 is 50 mg/mL, and the preset concentration of dalbavancin hydrochloride (calculated as dalbavancin free base) for Formulations 11-13 is 20 mg/mL; in TABLE 7, the preset concentration of dalbavancin hydrochloride (calculated as dalbavancin free base) for Formulations 14-17 is 20 mg/mL; in TABLE 9, the preset concentration of dalbavancin hydrochloride (calculated as dalbavancin free base) for Formulations 18-21 is 20 mg/mL; and in TABLE 10, the preset concentration of dalbavancin hydrochloride (calculated as dalbavancin free base) for Formulation 22 is 83.3 mg/mL.
¹After being formulated, the sample was stored at a low temperature of 2-8° C. After being stored at the low temperature of 2-8° C., the sample became milky white gel-like solid, which may be because the low temperature and high pH both cause decreased solubility of API and thus the precipitated, and other items were not detected.
²After being taken out from a stability chamber, the sample was stored at a low temperature of 2-8° C. After being stored at the low temperature of 2-8° C., the sample became milky white gel-like solid, which may be because the low temperature and high pH both cause decreased solubility of API and thus the precipitated, and other items were not detected.

Assay in TABLES 2, 5, 7, 9 and 10 refers to a ratio of the actually measured concentration of dalbavancin hydrochloride (calculated as dalbavancin free base) to a preset concentration of dalbavancin hydrochloride (calculated as dalbavancin free base), where in TABLE 2, the preset concentration of dalbavancin hydrochloride (calculated as dalbavancin free base) for Formulations 1-7 is 50 mg/mL; in TABLE 5, the preset concentration of dalbavancin hydrochloride (calculated as dalbavancin free base) for Formulations 8-10 is 50 mg/mL, and the preset concentration of dalbavancin hydrochloride (calculated as dalbavancin free base) for Formulations 11-13 is 20 mg/mL; in TABLE 7, the preset concentration of dalbavancin hydrochloride (calculated as dalbavancin free base) for Formulations 14-17 is 20 mg/mL; in TABLE 9, the preset concentration of dalbavancin hydrochloride (calculated as dalbavancin free base) for Formulations 18-21 is 20 mg/mL; and in TABLE 10, the preset concentration of dalbavancin hydrochloride (calculated as dalbavancin free base) for Formulation 22 is 83.3 mg/mL.

¹ After being formulated, the sample was stored at a low temperature of 2-8° C. After being stored at the low temperature of 2-8° C., the sample became milky white gel-like solid, which may be because the low temperature and high pH both cause decreased solubility of API and thus the precipitated, and other items were not detected.

² After being taken out from a stability chamber, the sample was stored at a low temperature of 2-8° C. After being stored at the low temperature of 2-8° C., the sample became milky white gel-like solid, which may be because the low temperature and high pH both cause decreased solubility of API and thus the precipitated, and other items were not detected.

It can be seen from TABLE 2 that when pH of the solvent is 3.5-5.5, the stabilities of the formulations can be improved, and when the pH of the solvent is 4.45-4.55, the effect is the most significant.

Chromatographic conditions for detecting the impurities are as shown in TABLE 3:

TABLE 3

| Chromatographic | Two ZhongpuRed RD-18, 150 mm × 2.1 mm, 1.8 μm | |
|---|---|---|
| Column | chromatographic columns were connected in | |
| | series with Agilent capillary tubes (0.17 | |
| | mm × 90 mm 1/16 male/male) for use | |
| UV Detector | Wavelength: 210 nm | |
| Flow rate | 0.15 | mL/min |
| Column | 55° | C. |
| temperature | | |
| Temperature of | 5° | C. |
| Sample Plate | | |
| Injection Volume | 2.0 | μL |
| Running Time | 120 | min |

| | |
|---|---|
| Needle Washing | 10% methanol |
| liquid | |

Instrument: Waters ultra-high performance liquid chromatograph.

Gradient elution is as shown in TABLE 4:

TABLE 4

| | Mobile Phase | Mobile Phase |
|---|---|---|
| Time (min) | A (%) | B (%) |
| 0 | 95 | 5 |
| 10 | 95 | 5 |
| 45 | 5 | 95 |
| 109 | 5 | 95 |
| 110 | 95 | 5 |
| 120 | 95 | 5 |

### Mobile Phase:

Buffer solution: 2.87 g of ammonium dihydrogen phosphate dissolved in 1000 mL of water, adjusted to pH of 6.0 with ammonia water, and filtered with a 0.22 μm filter membrane.

Mobile phase A: the buffer solution and acetonitrile mixed in a ratio of 80:20 (V/V), and degassed by means of sonication.

Mobile Phase B: the buffer solution and acetonitrile mixed in a ratio of 66:34 (V/V), and degassed by means of sonication.

In a chromatogram obtained after detection, except five peaks corresponding to A₀, A₁, B₀, B₁ and B₂ of dalbavancin hydrochloride, others were all recorded as impurities.

### Example 2: Effect of Solvent Composition on Stability of Dalbavancin Injection Formulation

The present example provides a preparation method for a dalbavancin injection formulation, mainly different from Example 1 in a different ratio of propylene glycol to water for injection in the solvent. Impurity contents of finished formulation products were measured when the preparation of the finished formulation products was completed and after the finished formulation products were placed at 40° C. for 10 days, to obtain stabilities of various formulations as shown in TABLE 5.

TABLE 5

| | | | Volume | | | | | | |
|---|---|---|---|---|---|---|---|---|---|
| | | | Fraction | | | | | | |
| | | | of | | | | | | |
| | | | Propylene | | | Related Substances (%) | | | |
| | | | Glycol in | | Assay | Impurity | Impurity | Impurity | Total |
| Formulation | | Appearance | Solvent % | pH | (%) | I | III | IV | Impurities |
| Formulation | Initial | Colorless | 20 | 4.52 | 101.1 | 0.856 | 0.050 | ND | 1.490 |
| 8 | | clear | | | | | | | |
| | | solution | | | | | | | |
| | 40° C. | Colorless | | 4.53 | 100.3 | 3.449 | 0.056 | 0.070 | 4.310 |
| | 10 | clear | | | | | | | |
| | days | solution | | | | | | | |
| Formulation | Initial | Colorless | 30 | 4.49 | 103.6 | 0.895 | 0.049 | ND | 1.532 |
| 9 | | clear | | | | | | | |
| | | solution | | | | | | | |
| | 40° C. | Colorless | | 4.52 | 99.8 | 3.037 | 0.055 | 0.075 | 3.961 |
| | 10 | clear | | | | | | | |
| | days | solution | | | | | | | |
| Formulation | Initial | Colorless | 40 | 4.52 | 100.2 | 0.826 | 0.052 | ND | 1.517 |
| 10 | | clear | | | | | | | |
| | | solution | | | | | | | |
| | 40° C. | Colorless | | 4.48 | 99.8 | 2.522 | 0.052 | 0.071 | 3.432 |
| | 10 | clear | | | | | | | |
| | days | solution | | | | | | | |
| Formulation | Initial | Colorless | 50 | 4.52 | 104.0 | 0.566 | 0.038 | 0.058 | 1.104 |
| 11 | | clear | | | | | | | |
| | | solution | | | | | | | |
| | 40° C. | N/A | | N/A | 103.7 | 2.094 | 0.042 | 0.120 | 3.034 |
| | 10 | | | | | | | | |
| | days | | | | | | | | |
| Formulation | Initial | Colorless | 75 | 4.34 | 96.8 | 0.736 | 0.039 | 0.058 | 1.278 |
| 12 | | clear | | | | | | | |
| | | solution | | | | | | | |
| | 40° C. | Colorless | | 4.42 | 90.3 | 5.025 | 0.037 | 0.113 | 6.196 |
| | 10 | clear | | | | | | | |
| | days | solution | | | | | | | |
| Formulation | Initial | Colorless | 100 | 4.32 | 95.0 | 0.969 | 0.037 | 0.057 | 1.523 |
| 13 | | clear | | | | | | | |
| | | solution | | | | | | | |
| | 40° C. | Colorless | 100 | 4.48 | 82.8 | 11.291 | 0.030 | 0.077 | 13.130 |
| | | clear | | | | | | | |
| | | solution | | | | | | | |

It can be seen from TABLE 5 that when the volume fraction of propylene glycol in the solvent is 30%-60%, the stability of the formulation can be improved, and when the volume fraction of propylene glycol in the solvent is 50%, the effect is the most significant.

It should be noted that volumes of hydrochloric acid and sodium hydroxide used for adjusting pH of the mixed solution are less than 1% of a volume of the dalbavancin injection formulation, and therefore the effect of the pH regulator on the volume ratio of propylene glycol to water for injection in the injection formulation can be ignored.

### Comparative Example 1: Effect of Solvent Type on Stability of Dalbavancin Injection Formulation

The present comparative example provides a preparation method for a dalbavancin injection formulation, mainly different from Example 1 in that: kinds of the solvents in the formulations are different. The solvents used for various formulations are listed in TABLE 6. Impurity contents of finished formulation products were measured when the preparation of the finished formulation products was completed and after the finished formulation products were placed at 40° C. for 10 days, to obtain stabilities of various formulations as shown in TABLE 7.

TABLE 6

| | | Volume Ratio | Bulk |
|---|---|---|---|
| | Formulation | of Solvents | Solution |
| Formulation | Material | for Preparation | pH |
| Formulation 14 | API | Anhydrous ethanol:water | 4.6 ± 0.1 |
| | | for injection = 1:1 | |
| Formulation 11 | API | Propylene glycol:water | 4.6 ± 0.1 |
| | | for injection = 1:1 | |
| Formulation 15 | API | Polyethylene glycol | 4.6 ± 0.1 |
| | | 300:water for | |
| | | injection = 1:1 | |
| Formulation 16 | API | Polyethylene glycol | 4.6 ± 0.1 |
| | | 400:water for | |
| | | injection = 1:1 | |
| Formulation 17 | API | N,N-Dimethylacetamide:water | 4.6 ± 0.1 |
| | | for injection = 1:1 | |

TABLE 7

| | | | | | Related Substances (%) | | | |
|---|---|---|---|---|---|---|---|---|
| | | | | Assay | Impurity | Impurity | Impurity | Total |
| Formulation | | Appearance | pH | (%) | I | III | IV | Impurities |
| Formulation | Initial | Colorless | 4.56 | 96.6 | 0.543 | 0.039 | 0.051 | 1.111 |
| 14 | | clear | | | | | | |
| | | solution | | | | | | |
| | 40° C. | Colorless | 4.56 | 94.3 | 2.519 | 0.038 | 0.126 | 3.493 |
| | 10 | clear | | | | | | |
| | days | solution | | | | | | |
| Formulation | Initial | Colorless | 4.52 | 104.0 | 0.566 | 0.038 | 0.058 | 1.104 |
| 11 | | clear | | | | | | |
| | | solution | | | | | | |
| | 40° C. | N/A | N/A | 103.7 | 2.094 | 0.042 | 0.120 | 3.034 |
| | 10 | | | | | | | |
| | days | | | | | | | |
| Formulation | Initial | Colorless | 4.35 | 108.4 | 0.574 | 0.041 | 0.056 | 1.129 |
| 15 | | clear | | | | | | |
| | | solution | | | | | | |
| | 40° C. | N/A | N/A | 106.1 | 2.965 | 0.043 | 0.088 | 3.690 |
| | 10 | | | | | | | |
| | days | | | | | | | |
| Formulation | Initial | Colorless | 4.27 | 106.8 | 0.577 | 0.041 | 0.051 | 1.406 |
| 16 | | clear | | | | | | |
| | | solution | | | | | | |
| | 40° C. | N/A | N/A | 103.9 | 2.962 | 0.043 | 0.146 | 4.106 |
| | 10 | | | | | | | |
| | days | | | | | | | |
| Formulation | Initial | Yellowish- | 4.59 | 39.0 | 0.078 | 0.067 | 0.172 | 85.501 |
| 17 | | brown clear | | | | | | |
| | | solution | | | | | | |

It can be seen from TABLE 6 and TABLE 7 that when the aqueous solution of propylene glycol is used as the solvent, the formulation has better stability, other solvents do not have an obvious effect of improving the stability of the formulation, and some even have an effect of reducing the stability of the formulation.

### Comparative Example 2: Effect of Stabilizer on Stability of Dalbavancin Injection Formulation

The present comparative example provides a preparation method for a dalbavancin injection formulation, mainly different from Example 1 in that water for injection is used as the solvent, and stabilizers are added into formulations, where compositions and use amounts of the stabilizers are listed in TABLE 8. Impurity contents of finished formulation products were measured when the preparation of the finished formulation products was completed and after the finished formulation products were placed at 40° C. for 10 days, to obtain stabilities of various formulations as shown in TABLE 9.

TABLE 8

| | | Compounding | Bulk Solution |
|---|---|---|---|
| Formulation | Formulation Material | Solvent | pH |
| Formulation | API (20 mg/mL, calculated as dalbavancin free | Water for | 4.6 ± 0.1 |
| 18 | base) + lactose monohydrate (5.16 mg/mL) + | injection | |
| | mannitol (5.16 mg/mL) | | |
| Formulation | API (20 mg/mL, calculated as dalbavancin free | Water for | 4.6 ± 0.1 |
| 19 | base) + lactose monohydrate (5.16 mg/mL) | injection | |
| Formulation | API (20 mg/mL, calculated as dalbavancin | Water for | 4.6 ± 0.1 |
| 20 | free base) | injection | |
| Formulation | API (20 mg/mL, calculated as dalbavancin free | Water for | 4.6 ± 0.1 |
| 21 | base) + lactose monohydrate (5.16 mg/mL) + | injection | |
| | mannitol (5.16 mg/mL) + glucose (50 mg/mL) | | |

TABLE 9

| | | | | | Related Substances (%) | | | |
|---|---|---|---|---|---|---|---|---|
| | | | | Assay | Impurity | Impurity | Impurity | Total |
| Formulation | | Appearance | pH | (%) | I | III | IV | Impurities |
| Formulation | Initial | Colorless | 4.55 | 100.3 | 0.794 | 0.049 | 0.030 | 1.403 |
| 18 | | clear | | | | | | |
| | | solution | | | | | | |
| | 40° C. | Colorless | 4.56 | 97.3 | 3.353 | 0.071 | 0.029 | 4.226 |
| | 10 | clear | | | | | | |
| | days | solution | | | | | | |
| Formulation | Initial | Colorless | 4.54 | 100.1 | 0.791 | 0.050 | 0.021 | 1.376 |
| 19 | | clear | | | | | | |
| | | solution | | | | | | |
| | 40° C. | Colorless | 4.51 | 97.1 | 3.240 | 0.068 | 0.027 | 4.102 |
| | 10 | clear | | | | | | |
| | days | solution | | | | | | |
| Formulation | Initial | Colorless | 4.51 | 100.1 | 0.816 | 0.041 | 0.022 | 1.382 |
| 20 | | clear | | | | | | |
| | | solution | | | | | | |
| | 40° C. | Colorless | 4.49 | 97.0 | 3.148 | 0.066 | 0.024 | 3.999 |
| | 10 | clear | | | | | | |
| | days | solution | | | | | | |
| Formulation | Initial | Colorless | 4.49 | 103.1 | 0.498 | 0.037 | 0.046 | 1.034 |
| 21 | | clear | | | | | | |
| | | solution | | | | | | |
| | 40° C. | Colorless | 4.60 | 99.3 | 3.200 | 0.055 | 0.105 | 3.937 |
| | 10 | clear | | | | | | |
| | days | solution | | | | | | |

According to TABLE 8 and TABLE 9, it can be seen that adding the stabilizer (lactose monohydrate, mannitol or glucose) to the formulation does not significantly improve the stability of the product, and may even reduce the stability of the product.

### Example 3: Effect of API Concentration on Stability of Dalbavancin Injection Formulation

The present example provides a preparation method of dalbavancin injection formulation, wherein the concentration of API (active pharmaceutical ingredient) in solution is higher than that in Example 1. This method specifically included the followings.

Into a compounding tank, about 400 mL of water for injection was added, with a solution temperature kept at 25° C., a batch required quantity of propylene glycol was added (the volume ratio of propylene glycol to water for injection is 4:6), mixture was well stirred, then a batch required quantity (theoretical required quantity: 83.3 g, calculated as dalbavancin free base) of dalbavancin hydrochloride was added, and stirred to be dissolved to obtain a clear solution.

The pH of bulk solution was adjusted to a preset value 4.5±0.1 using a pH regulator (sodium hydroxide), and then the bulk solution was made up to 1000 mL with water for injection.

The bulk solution was filtered through a sterilization filter. About 6.4 mL of the bulk solution was filled into a 6 mL clear vial which was then full-stoppered with a 13 mm stopper and capped with an aluminum cap to obtain the finished formulation product, i.e., formulation 22. Impurity contents of the finished formulation product were measured when the preparation of the finished formulation product was completed and after the finished formulation product was placed at 40° C. for 10 days, to obtain stabilities thereof as shown in TABLE 10 below.

TABLE 10

| | | | | | Related Substances (%) | | | |
|---|---|---|---|---|---|---|---|---|
| | | | | Assay | Impurity | Impurity | Impurity | Total |
| Formulation | | Appearance | pH | (%) | I | III | IV | Impurities |
| Formulation | Initial | Colorless | 4.56 | 104.2 | 1.054 | 0.035 | 0.065 | 1.587 |
| 22 | | clear | | | | | | |
| | | solution | | | | | | |
| | 40° C. | Colorless | 4.57 | 100.8 | 3.134 | 0.040 | 0.083 | 3.813 |
| | 10 | clear | | | | | | |
| | days | solution | | | | | | |

It can be seen from the above results that after the formulation 22 is placed at 40° C. for 10 days, the total impurities thereof are less than 5.0%, the increase in the content of impurity I is less than or equal to 2.5% (≤2.5%), and the content of the main drug is decreased by 5.0% or less (≤5.0%), indicating that the formulation has good stability.

Besides, in order to investigate admixture compatibility stability of the formulation of the present disclosure in clinical use, the admixture compatibility stability of the formulation 22 was studied. A method for testing the admixture compatibility stability included: diluting the formulation 22 with water for injection to a 20 mg/mL solution, and then diluting the same using only a 5% Dextrose Injection to solutions at final concentrations of 1 mg/mL and 5 mg/mL respectively, and investigating the stabilities of the resulting 1 mg/mL and 5 mg/mL solutions at 25° C. Results are as shown in TABLE 11 below:

TABLE 11

| | | Formulation 22 | | | | | | | | | | | |
|---|---|---|---|---|---|---|---|---|---|---|---|---|---|
| | | Concentration: 1 mg/mL | | | | | | Concentration: 5 mg/mL | | | | | |
| Detection Item | | 0 d | 1 d | 2 d | 3 d | 9 d | 14 d | 0 d | 1 d | 2 d | 3 d | 9 d | 14 d |
| Solution | | Color- | Color- | Color- | Color- | Color- | Color- | Color- | Color- | Color- | Color- | Color- | Color- |
| Appearance | | less | less | less | less | less | less | less | less | less | less | less | less |
| | | clear | clear | clear | clear | clear | clear | clear | clear | clear | clear | clear | clear |
| | | solution | solution | solution | solution | solution | solution | solution | solution | solution | solution | solution | solution |
| pH | | 3.93 | 3.85 | 3.86 | 3.79 | 3.88 | 3.93 | 3.61 | 3.59 | 3.58 | 3.54 | 3.63 | 3.55 |
| Osmolality | | 312 | 303 | 334 | 322 | 319 | 320 | 510 | 497 | 547 | 518 | 514 | 530 |
| (mOsmol/kg) | | | | | | | | | | | | | |
| Assay (%) | | 104.7 | 105.6 | 105.3 | 105.3 | 103.9 | 104.8 | 103.7 | 103.2 | 104.8 | 101.8 | 101.9 | 102.8 |
| Related | Impurity | 1.071 | 1.072 | 1.103 | 1.132 | 1.275 | 1.357 | 1.064 | 1.109 | 1.144 | 1.175 | 1.373 | 1.529 |
| Substances | I | | | | | | | | | | | | |
| (%) | Impurity | 0.036 | 0.035 | 0.034 | 0.038 | 0.035 | 0.037 | 0.034 | 0.034 | 0.039 | 0.032 | 0.034 | 0.037 |
| | III | | | | | | | | | | | | |
| | Impurity | 0.066 | 0.063 | 0.075 | 0.070 | 0.054 | 0.067 | 0.067 | 0.069 | 0.073 | 0.078 | 0.059 | 0.056 |
| | IV | | | | | | | | | | | | |
| | Total | 1.610 | 1.619 | 1.676 | 1.710 | 1.766 | 1.881 | 1.623 | 1.658 | 1.735 | 1.765 | 1.838 | 2.008 |
| | Impurities | | | | | | | | | | | | |

It can be seen from the above results that the diluted solutions prepared from the formulation 22 obtained in the present disclosure can be stable for at least 14 days under a condition of 25° C., that is, it has good admixture compatibility stability in clinical use.

The above are merely preferred examples of the present disclosure, but are not intended to limit the present disclosure. For those skilled in the art, various modifications and changes could be made to the present disclosure. Any amendments, equivalent replacements, improvements and so on, made within the spirit and principle of the present disclosure, should be covered within the scope of protection of the present disclosure.

What is claimed is:

1. A method of using an aqueous solution of propylene glycol, comprising using the aqueous solution of propylene glycol to inhibit degradation of dalbavancin hydrochloride.

2. The method of using an aqueous solution of propylene glycol according to claim 1, wherein in the aqueous solution of propylene glycol, a volume ratio of propylene glycol to water for injection is 2:8-6:4.

3. The method of using an aqueous solution of propylene glycol according to claim 2, wherein in the aqueous solution of propylene glycol, the volume ratio of propylene glycol to water for injection is 4:6-6:4.

4. The method of using an aqueous solution of propylene glycol according to claim 3, wherein in the aqueous solution of propylene glycol, the volume ratio of propylene glycol to water for injection is 5.5:4.5-4.5:5.5.

5. The method of using an aqueous solution of propylene glycol according to claim 3, wherein in the aqueous solution of propylene glycol, the volume ratio of propylene glycol to water for injection is 4:6.

6. A dalbavancin injection formulation, comprising dalbavancin hydrochloride and an aqueous solution of propylene glycol, wherein the aqueous solution of propylene glycol can inhibit degradation of dalbavancin hydrochloride, and a concentration of dalbavancin hydrochloride is 20-120 mg/mL, calculated as dalbavancin free base.

7. The dalbavancin injection formulation according to claim 6, wherein the concentration of dalbavancin hydrochloride is 20-60 mg/mL, calculated as dalbavancin free base.

8. The dalbavancin injection formulation according to claim 6, wherein the concentration of dalbavancin hydrochloride is 60-90 mg/mL, calculated as dalbavancin free base.

9. The dalbavancin injection formulation according to claim 8, wherein the concentration of dalbavancin hydrochloride is 80-85 mg/mL, calculated as dalbavancin free base.

10. The dalbavancin injection formulation according to claim 6, wherein pH of the dalbavancin injection formulation is 4.0-5.0.

11. The dalbavancin injection formulation according to claim 10, wherein the pH of the dalbavancin injection formulation is 4.4-4.6.

12. The dalbavancin injection formulation according to claim 11, wherein the pH of the dalbavancin injection formulation is 4.45-4.55.

13. The dalbavancin injection formulation according to claim 3, wherein after the dalbavancin injection formulation is placed at 40° C. for 10 days, the dalbavancin injection formulation satisfies at least one of:
total impurities are less than 5.0%;
an increase in a content of an impurity as shown by Formula I is less than or equal to 2.5%, wherein a compound as shown by Formula I is of a structure as follows: 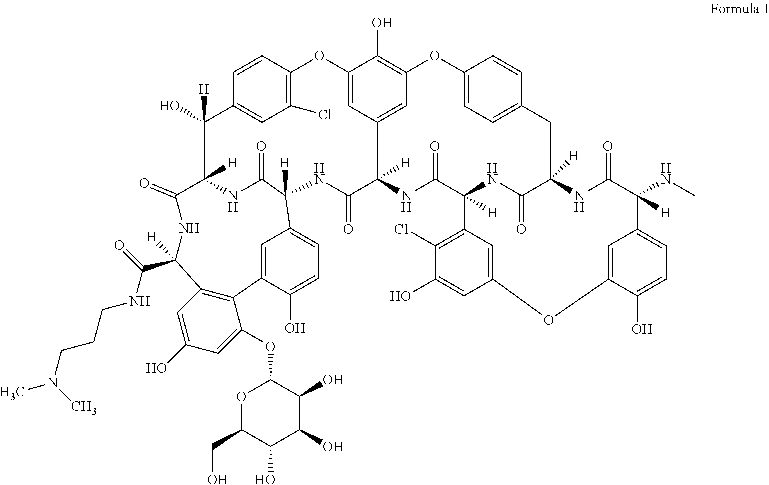
a content of a main drug is decreased by 5.0% or less, wherein the main drug is dalbavancin hydrochloride, comprising compounds as shown by Formula II: 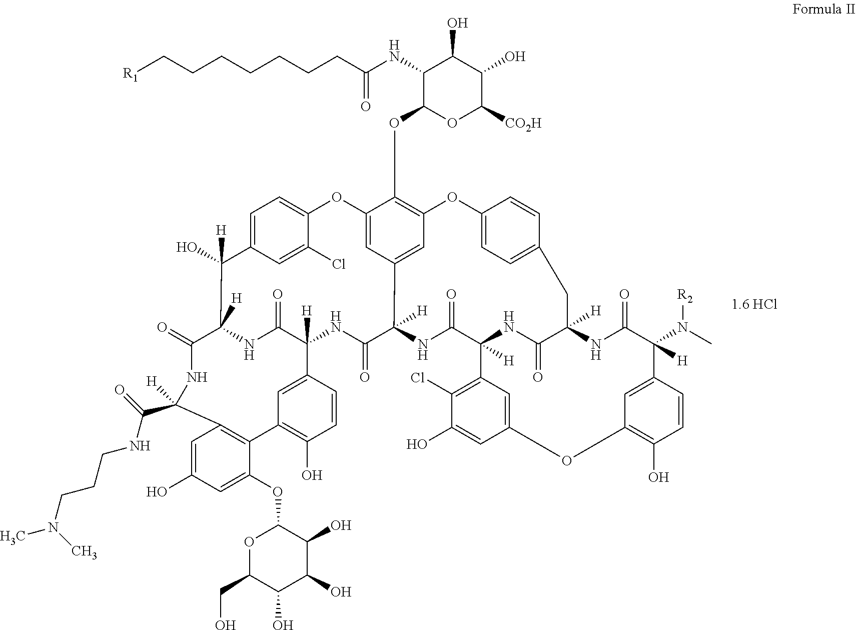
the main drug comprises A₀, A₁, B₀, B₁ and B₂, wherein molecular formulas of A₀, A₁, B₀, B₁ and B₂ and molecular weights of corresponding anhydrous free bases are as shown in TABLE 1:
TABLE 1

| | | | Molecular | Molecular |
|---|---|---|---|---|
| Dalbavancin | R₁ | R₂ | Formula | Weight |
| A₀ | CH(CH₃)₂ | H | C₈₇H₉₈N₁₀O₂₈Cl₂ · 1.6 HCl | 1802.7 |
| A₁ | CH₂CH₂CH₃ | H | C₈₇H₉₈N₁₀O₂₈Cl₂ · 1.6 HCl | 1802.7 |
| B₀ | CH₂CH(CH₃)₂ | H | C₈₈H₁₀₀N₁₀O₂₈Cl₂ · 1.6 HCl | 1816.7 |
| B₁ | CH₂CH₂CH₂CH₃ | H | C₈₈H₁₀₀N₁₀O₂₈Cl₂ · 1.6 HCl | 1816.7 |
| B₂ | CH₂CH(CH₃)₂ | CH₃ | C₈₉H₁₀₂N₁₀O₂₈Cl₂ · 1.6 HCl | 1830.7 |

14. A preparation method for the dalbavancin injection formulation according to claim 6, comprising:
mixing the aqueous solution of propylene glycol with dalbavancin hydrochloride to obtain a clear mixed solution, and
adjusting pH of the mixed solution using a pH regulator to obtain an in-process product bulk solution of dalbavancin injection.

15. The preparation method for the dalbavancin injection formulation according to claim 14, further comprising:
sterilization filtering, filling, stoppering and capping the in-process product bulk solution of dalbavancin injection, to obtain the dalbavancin injection formulation packaged.

16. The preparation method for the dalbavancin injection formulation according to claim 14, wherein the pH regulator is at least one selected from the group consisting of hydrochloric acid and sodium hydroxide; and/or,
the mixing and adjusting pH of the mixed solution are both performed under a condition of 15-30° C.

17. The preparation method for the dalbavancin injection formulation according to claim 14, wherein the mixing the aqueous solution of propylene glycol with dalbavancin hydrochloride to obtain the clear mixed solution comprises:
adding about 40% of a batch size of water for injection into a compounding tank, with a solution temperature kept at 15-30° C., adding a batch required quantity of propylene glycol, and stirring well; and
adding a batch required quantity of dalbavancin hydrochloride, and stirring until dalbavancin hydrochloride is dissolved to obtain the clear solution.

18. The preparation method for the dalbavancin injection formulation according to claim 14, wherein the adjusting pH of the mixed solution using the pH regulator to obtain the in-process product bulk solution of dalbavancin injection comprises:
adjusting pH of the mixed solution with the pH regulator to 3.5-5.5, and then adding the balance amount of water for injection to volume.

19. A method of using the dalbavancin injection formulation according to claim 6, comprising using the dalbavancin injection formulation to prepare a drug for treating bacterial infections.

20. The method of using the dalbavancin injection formulation according to claim 19, wherein the bacterial infections comprise acute bacterial skin and skin structure infections caused by designated susceptible strains of Gram-positive microorganisms.
